(19) United States
(12) Patent Application Publication
(10) Pub. No.: US 2014/0072534 A1
(43) Pub. Date: Mar. 13, 2014

(54) NOVEL THERAPY FOR MULTIPLE SCLEROSIS USING VITAMIN D AND GUT BACTERIA

(71) Applicant: California Institute of Technology, Pasadena, CA (US)

(72) Inventors: Sarkis K. Mazmanian, Porter Ranch, CA (US); Yunkyung Lee, Los Angeles, CA (US)

(73) Assignee: California Institute of Technology, Pasadena, CA (US)

(21) Appl. No.: 14/015,769

(22) Filed: Aug. 30, 2013

Related U.S. Application Data
(60) Provisional application No. 61/695,203, filed on Aug. 30, 2012.

(51) Int. Cl. A61K 35/74; A61K 31/593; A61K 31/715

(52) U.S. Cl. CPC A61K 35/74; A61K 31/715; A61K 31/593
USPC 424/93.4; 514/23

(57) ABSTRACT

A method for treating multiple sclerosis in a subject in need of such treatment is provided. The method includes administering a) polysaccharide A and b) vitamin D or a vitamin D metabolite to the subject in an amount effective to treat multiple sclerosis. Also provided is a method for increasing T_{REG} cells in a subject. The method includes administering a) polysaccharide A and b) vitamin D or a vitamin D metabolite to the subject in an amount effective to increase the number of T_{REG} cells in the central nervous system of the subject.

### BACKGROUND

1. Field of the Invention

The invention relates to methods and compositions for treating multiple sclerosis.

2. Related Art

Multiple sclerosis (MS) is an autoimmune disease that affects the central nervous system (CNS) [12]. Common early symptoms include numbness, pain, burning, and itching. Cognitive problems include memory disturbances, decreased judgment, and inattention. Disease involves autoreactive T cells, B cells and other immune cells that infiltrate the CNS and attack the myelin sheath, the protective coating of nerves [13]. Though significant clinical and scientific efforts have been expended, the cause(s) of MS remain largely unknown, and safe and effective treatments are still needed. MS appears to involve genetic as well as environmental factors. Genome wide association studies have revealed polymorphisms in genes that encode for MHC molecules, cytokines and their receptors, and genes associated with other autoimmune disorders [14]. However, low concordance rates in monozygotic twins [15] that do not appear to be mediated solely by genomic differences [16], and the rapid rise in genetically stable populations [17] indicate that environmental factors likely contribute to MS.

Experimental autoimmune encephalomyelitis (EAE) is an animal model that reproduces many of the features of MS [18]. EAE can be induced by immunization with CNS antigens, including myelin basic protein (MBP), myelin oligodendrocyte glycoprotein (MOG) or proteolipid protein (PLP). Peripheral immune activation of autoreactive T cells is believed to be a critical initiating step in EAE, and bacterial and viral infections have been reported to possibly promote MS [19]. Cross-reactivity via molecular mimicry between pathogen and self-proteins are speculated as a trigger; but studies have failed to conclusively identify infectious agents as a cause for MS.

### SUMMARY

In one aspect, a method for treating multiple sclerosis in a subject in need of such treatment is provided. The method includes administering a) polysaccharide A and b) vitamin D or a vitamin D metabolite to the subject in an amount effective to treat multiple sclerosis.

In another aspect, a method for increasing T_{REG} cells in a subject is provided. The method includes administering a) polysaccharide A and b) vitamin D or a vitamin D metabolite to the subject in an amount effective to increase the number of T_{REG} cells in the central nervous system of the subject.

In these methods, polysaccharide A can be administered by administering B. fragilis cells containing polysaccharide A, or by administering a cell extract, a membrane preparation, or a vesicle preparation containing polysaccharide A, or any combination thereof. In some cases, polysaccharide A can be purified or isolated before administering.

Also, in either method, the vitamin D metabolite can be 25-hydroxyvitamin D₃ or 1,25-dihydroxyvitamin D₃, the subject can be a mouse or a human, and/or polysaccharide A and vitamin D or a vitamin D metabolite can be orally administered.

### BRIEF DESCRIPTION OF THE DRAWINGS

For a more complete understanding of the present invention, reference is now made to the following descriptions taken in conjunction with the accompanying drawings, in which:

FIGS. 1A-1B. Drawing of a proposed model for microbiome-mediated effects on vitamin D and T cell responses during EAE. (FIG. 1A) Inactive vitamin D (25D) is converted to active vitamin D (1,25D) by Cyp27b1. Active 1,25D binds the vitamin D receptor (VDR) in T cells to promote T_{REG} development and function and suppress T_{H}17 cell responses. (FIG. 1B) To summarize preliminary data, segmented filamentous bacteria (SFB) lower VDR expression levels in gut during colonization, skewing T cell responses away from T_{REGS} and toward T_{H}17 cells. SFB promote EAE through induction of T_{H}17 cells. Germ-free mice have reduced levels of active 1,25D. PSA from B. fragilis increases Cyp27b1 and VDR expression, resulting in increased levels of 1,25D. PSA induces T_{REG} cells in the CNS during protection from EAE

FIGS. 2A-2D. Graphs showing germ-free (GF) mice are attenuated in EAE. Female C57Bl/6 mice were immunized subcutaneously (s.c.) with 150 μg MOG₃₅₋₅₅ peptide with 200 μg of Complete Freund's adjuvant (CFA). On the same day and 2 days later, mice were injected intraperitoneally (i.p.) with 150 ng/mouse of pertussis toxin. (FIG. 2A) Clinical EAE scores of SPF (dark symbols) and GF (light symbols). Data are representative of three independent experiments. (FIG. 2B) Proportions of SPF and GF mice that develop various degrees of EAE, as assessed by clinical score [18]. Results are combined from 4 experiments, with a total of 20 for SPF and 22 for GF mice. (FIG. 2C) IFNγ and IL-17A cytokine ELISA analysis of cells harvested from draining lymph nodes of SPF and GF mice 8 days after immunization with MOG/CFA and cultured for 3 days in vitro with MOG peptide. (FIG. 2D) CD4+CD25+Foxp3+ T cells at day 15 post-immunization. Cells were cultured for 3 days with MOG peptide, and analyzed by flow cytometry. Data from Lee et al., PNAS, 2011 [2].

FIGS. 3A-3C. Graphs and plots showing gut colonization with SFB promotes EAE. (FIG. 3A) EAE scores of SPF, GF and GF-SFB colonized mice. GF mice were colonized with SFB for 3 weeks prior to MOG/CFA immunization (GF-SFB). Data are representative of two experiments. (FIG. 3B) FC plots of IL-17A and IFNγ-producing CD4+ T cells from gut and spinal cords of SPF, GF and GF-SFB colonized mice at day 15 after EAE induction. Numbers in each quadrant indicate percentage of cytokine positive cells. (FIG. 3C) IL-17A and Foxp3 producing CD4+ T cells from spinal cords and small intestines of SPF, GF and GF-SFB colonized mice at day 15 after EAE induction. Numbers in each quadrant indicate percentage of positive cells. Data from Lee et al., PNAS, 2011 [2].

FIGS. 4A-4G. Graphs showing effects of the microbiota on vitamin D metabolism in vivo. (FIG. 4A) qRT-PCR of mRNA from small intestine (SI) and kidney for Cyp27b1 shows decreased expression in GF mice. NS, not significant. *p<0.01 (FIG. 4B) GF animals show reduced serum levels of 1,25D. *p<0.001, but not 25D, suggesting a defect in conversion to active vitamin D (FIG. 4C) Colonic Cyp27b1 mRNA in SPF, GF, and mice mono-colonized with B. fragilis (B.f.) or B. fragilis lacking PSA (ΔPSA). B. fragilis induces Cyp27b1 in a PSA-dependent manner. *p<0.01 (FIG. 4D) Serum 1,25D levels in GF mice, or GF mice fed 50 μg PSA 3× per week for 2 weeks. *p<0.005. (FIG. 4E) mRNA levels of VDR in colonic tissue of SPF, GF and GF mice fed PSA. PSA induces VDR, suggesting it sensitizes mice to vitamin D. (FIG. 4F) Mice on vitamin D sufficient (Suf) or deficient (Def) chow for 6 weeks were fed PSA for 2 weeks, and Cyp27b1 expression was analyzed by qRT-PCR. PSA induces increased Cyp21B1 expression in the colon of vitamin D deficient mice. (FIG. 4G) GF mice were compared to SFB mono-colonized and SPF mice for expression of Cyp27b1 and VDR in the SI. While Cyp27b1 levels are not affected, SFB lowers VDR expression, suggesting it promotes insensitivity to 1,25D.

FIGS. 5A-5C. Graphs and plots showing vitamin D augments the ability of PSA to promote T_{REGS}. (FIG. 5A) Mouse BMDCs were stimulated with PSA (or PBS), and Cyp27b1 transcript levels were measured by qRT-PCR. PSA induces the expression of Cyp27b1 from WT, but not TLR2−/− DCs. (FIG. 5B) Mouse DCs were treated with PSA, washed and co-cultured with naïve CD4+ T cells. After 4 days, PSA increased Foxp3+T_{REGS} (see % in red box). The vitamin D metabolite 25D augmented PSA-induced T_{REGS}. (FIG. 5C) Human monocytes were treated with PSA or vehicle, washed and co-cultured with CD4+ T cells from PBMCs of the same donor. The MFI for Foxp3 among CD4+CD25+ T cells is shown, with or without TGFβ.

FIG. 6. Graph showing a link between PSA and vitamin D during EAE. Clinical EAE scores of indicated mice during EAE development. 3 week old female C57BL/6 mice were either raised on a normal diet (Nor) or vitamin D-deficient diet (Def) for 6 weeks. PSA (100 μg/dose) was given orally 3×/week throughout the experiment.

FIG. 7. Graph showing dendritic cells from GF animals are defective in inducing T_{H}17 responses. CD4+ T cells from MOG-Tg mice were cultured with mesenteric lymph node (MLN) DCs from SPF or GF mice, with or without MOG peptide and IL-2. At day 3 and day 5 of culture, IL-17A was measured by ELISA. *p<0.05. Similar results were observed for T_{H}1 cells. ND: not detectable. Data from Lee et al., PNAS, 2011 [2].

FIG. 8. Drawing of a model for interaction between PSA and the vitamin system. PSA increases Cyp27b1 expression in DCs, leading to conversion of inactive 25D to active 1,25D. 1,25D is sensed by VDR in T cells to promote IL-10 production from CD4+Foxp3+ T_{REGS}. IL-10 producing T_{REG} cells are required for protection from EAE [9,22].

### DETAILED DESCRIPTION

The following is incorporated by reference herein: Provisional Patent Application No. 61/695,203, filed on Aug. 30, 2012.

As used herein, polysaccharide A (or PSA, or PSA ligand) refers to a molecule produced by the PSA locus of Bacteroides fragilis and derivatives thereof which include but are not limited to polymers of the repeating unit {→3) α-d-AAT Galp(1→4)-[β-d-Galf(1→3)]α-d-GalpNAc(1→3)-[4,6-pyruvate]-β-d-Galp(1→}, where AATGal is acetamido-amino-2,4,6-trideoxygalactose, and the galactopyranosyl residue is modified by a pyruvate substituent spanning O-4 and O-6. The term “derivative” as used herein with reference to a first polysaccharide (e.g., PSA), indicates a second polysaccharide that is structurally related to the first polysaccharide and is derivable from the first polysaccharide by a modification that introduces a feature that is not present in the first polysaccharide while retaining functional properties of the first polysaccharide. Accordingly, a derivative polysaccharide of PSA, usually differs from the original polysaccharide by modification of the repeating units or of the saccharidic component of one or more of the repeating units that might or might not be associated with an additional function not present in the original polysaccharide. A derivative polysaccharide of PSA retains however one or more functional activities that are herein described in connection with PSA in association with the anti-inflammatory activity of PSA.

Vitamin D refers to any one or a combination of a group of fat-soluble prohormones (D1-D5; 25 D, 1,25 D, see below), which encourages the absorption and metabolism of calcium and phosphorous. Five forms of vitamin D have been discovered, vitamin D₁, D₂, D₃, D₄, D₅. The two forms that seem to matter to humans the most are vitamins D₂ (ergocalciferol) and D₃ (cholecalciferol). Vitamin D for humans is obtained from sun exposure, food and supplements. It is biologically inert and has to undergo two hydroxylation reactions to become active in the body. Metabolites of vitamin D include 25-hydroxyvitamin D₃, and 1,25-dihydroxycholecalciferol or 1,25-dihydroxyvitamin D₃ (1,25D), which is considered the active form of vitamin D.

1,25 D is derived from its precursor 25-hydroxyvitamin-D₃ (25D) by the enzyme 1α-hydroxylase (“CYP27B1”) encoded by the CYP27B1 gene, (NG_{—}007076.1 Homo Sapiens) CYP27B1.

Polysaccharide A and/or vitamin D compounds can be formulated as pharmaceutical compositions which include pharmaceutically acceptable or appropriate carriers. Such carriers can be, but are not limited to, organic or inorganic, solid or liquid excipient which is suitable for the selected mode of application such as oral application or injection, and administered in the form of a conventional pharmaceutical preparation. Such preparation includes solid such as tablets, granules, powders, capsules, and liquid such as solution, emulsion, suspension and the like. Said carrier includes starch, lactose, glucose, sucrose, dextrine, cellulose, paraffin, fatty acid glyceride, water, alcohol, gum arabic and the like. If necessary, auxiliary, stabilizer, emulsifier, lubricant, binder, pH adjustor controller, isotonic agent and other conventional additives may be added. The compositions can also be formulated with dispersing and surface active agents, binders, and lubricants. One skilled in this art may further formulate the compounds in an appropriate manner, and in accordance with accepted practices, such as those disclosed in Remington's Pharmaceutical Sciences, Gennaro, Ed., Mack Publishing Co., Easton, Pa. 1990. The amount of active compound administered will be dependent on the subject being treated, the subject's weight, the manner of administration and the judgment of the prescribing physician.

Routes of administration will vary with the make up of the composition and can include, for example, intradermal, transdermal, parenteral, intravenous, intramuscular, intranasal, subcutaneous, percutaneous, intraperitoneal, perfusion, lavage, direct injection, and oral administration.

In the method for treating multiple sclerosis, an amount effective to treat multiple sclerosis is an amount that results in a therapeutic benefit. A therapeutic benefit refers to a treatment that promotes or enhances the well-being of the subject with respect to the treated condition, which includes, but is not limited to, extension of the subject's life by any period of time, a decrease in pain to the subject that can be attributed to the subject's condition, a decrease in the severity of the disease, an increase in the therapeutic effect of a therapeutic agent, an improvement in the prognosis of the condition or disease, a decrease in the amount or frequency of administration of a therapeutic agent, or an alteration in the treatment regimen of the subject. For example, a therapeutic benefit can include a reduction in the severity or duration in any one or more clinical MS symptoms, such as, but not limited to fatigue; visual disorders; numbness; dizziness/vertigo; bladder and bowel dysfunction; weakness; tremor; impaired mobility; sexual dysfunction; slurred speech; spasticity (leg stiffness); swallowing disorders; chronic aching pain; depression; mild cognitive and memory difficulties.

In addition to PSA and vitamin D or vitamin D metabolites, a treatment can also include one or a combination of medicaments/treatments known to be useful in the treatment of MS such as, but not limited to, Avonex®, Betaseron®, and Copaxone®. Rebif®; Extavia® Novantrone® (mitoxantrone); Tysabri® (natalizumab), and Gilenya® (fingolimod). Other drugs include intravenous immunoglobulin (IVIg) therapy, methotrexate, azathioprine (Imuran®), and cyclophosphamide (Cytoxan®); corticosteroids; cytoxan® (cyclophosphamide); Imuran® (azathioprine); methotrexate; plasma exchange; pulse solu-medrol® (IV methylprednisolone); prednisone; Decadron® (dexamethasone); Medrol® (oral methylprednisolone); Plasmapheresis (plasma exchange); intravenous immunoglobulin (IVIg) therapy.

B. fragilis strain NCTC 9343 can be obtained from the American Type Culture Collection (Manassas, Va.). Bacteria can be grown, for example, in a rich medium containing 37 g BHI (brain heart infusion), 0.5 μg/100 ml Hemin, and 0.5 μg/ml Vitamin K in 1 L ddH2O or a customized minimum medium (MM), which contains 8 g Glucose, 1% FBS (fetal bovine serum), 0.5 μg/ml Hemin, and 0.5 μg/ml Vitamin K in 1 L of RPMI medium.

Bacterial cell extracts can be obtained using various chemical, immunological, biochemical or physical procedures known to those of skill in the art, including but not limited to, precipitation, centrifugation, filtering, column chromatography, and detergent lysis. An extract can contain soluble and/or membrane fractions.

Vesicles containing polysaccharide A can be prepared from B. fragilis. For example, Percoll discontinuous density-gradient centrifugation can be used for EDL (electron dense layer) isolation of B. fragilis (Patrick S, Reid J H. (1983) J Med Microbiol. 16(2): 239-41). Briefly, a 20%, 40%, 60%, 80% Percoll gradient (diluted with PBS) is created in a 14 ml test tube (2 ml for each layer). Then a B. fragilis culture resuspended in PBS is carefully added on top of the 20% Percoll layer. Subsequently, the gradient is centrifuged at 800 g for 20 min at RT. EDL-enriched bacteria can be recovered from the 40%-60% interface of the gradient after the separation. Outer membrane vesicles (OMV)s can be prepared from EDL-enriched B. fragilis grown in customized MM. OMVs are recovered from the bacteria-free supernatant of the culture by centrifugation at 22,000 g for 2 hrs at 4 C and further washed twice with PBS and filtered through 0.45 μm spin columns.

Purified PSA can be prepared as follows. Briefly, Bacteroides fragilis is grown, cells harvested, and phenol/chloroform extracts prepared. Proteins, nucleic acids are digested with enzymes, and the remaining material which is highly enriched for PSA is subjected to size exclusion chromatography to purify PSA. Purified PSA can also be prepared by published methods [75].

To assess EAE in mice, mice can be evaluated daily for signs of disease. The clinical course of the EAE can be scored according to the severity as follows: 0=no obvious signs of disease, 0.5=partial tail weakness (cannot raise tail against gravity), 1=limp tail, 1.5=limp tail and hindlimb weakness, 2=limp tail and impairment in righting reflex, 2.5=limp tail and hindlimb paresis (as determined by gait abnormality), 3=bilateral hindlimb paralysis, 3.5=bilateral hindlimb paralysis and forelimb paresis (as determined by in ability of the mouse to strongly grasp cage bars with the fore paws when placed on top), 4=moribund, 5=dead. Mice can be followed for a total of 30 days, at which point mice can be euthanized regardless of disease status. Reduced symptoms of EAE can be evaluated by comparing clinical scores.

The present invention may be better understood by referring to the accompanying examples, which are intended for illustration purposes only and should not in any sense be construed as limiting the scope of the invention.

### EXAMPLE 1

Mammals are colonized with 100 trillion indigenous bacteria that have profound contributions to human health [20]. The gastrointestinal tract harbors the greatest numbers and complexity of microorganisms, known as the gut microbiota, which regulates human nutrition, metabolism and immune function [21]. The inventors have shown that the microbiota mediates the development of the mammalian immune system [3]. Most intriguingly, the inventors and other groups have recently linked the gut microbiota to EAE [2,6,7]. The human commensal, Bacteroides fragilis, produces an immunomodulatory molecule (named polysaccharide A; PSA) that prevents and treats colitis in mice [4,5,22]. Furthermore, Kasper and co-workers have shown that PSA ameliorates and treats EAE [6,8]. PSA from B. fragilis suppresses uncontrolled immune activation in both experimental colitis and EAE, and thus represents a potential therapy for autoimmune disease [23].

Time magazine listed the “Benefits of Vitamin D” as a Top 10 Medical Breakthrough in 2007. Two pivotal concepts are central to a new perspective on vitamin D: 1) sub-optimal vitamin D status or vitamin D insufficiency is a prevalent health problem in Western societies; and 2) vitamin D is a potent modulator of innate and adaptive immunity. Serum vitamin D deficiency or lower dietary vitamin D intake is associated with MS [24,25]. Studies have reported a strong inverse correlation between exposure to sunlight (UV radiation produces vitamin D in the skin) and incidence of MS (reviewed in [26,27,28,29]. Randomized placebo-controlled trials in humans await to be done. Nevertheless, it is promising that studies in EAE have shown increased disease under conditions of dietary vitamin D restriction [30], while EAE mice treated with active 1,25D are protected from disease [31,32].

Uncontrolled immune activation leads to pathologies such as inflammatory bowel disease (IBD), type 1 diabetes (T1D), rheumatoid arthritis (RA) and MS. A primary mechanism to prevent deleterious immune responses is mediated by regulatory T cells (T_{REGS}) [33]. Various subsets of CD4+ T_{REG} cells control organ-specific autoimmunity, and T_{REGS} appear to be defective in MS patients [34]. In contrast, effector CD4+ T cells (T_{EFF}), such as T_{H}1 and T_{H}17 cells drive inflammation and autoimmune disease. T_{EFF} cells are believed to be pathogenic in EAE and MS, but how they become dysregulated during disease remains an intense area of investigation. Recent studies now reveal that the microbiota regulates CD4+ T cell development and function [35]. PSA from B. fragilis potently induces the development of interleukin-10 (IL-10) producing Foxp3+T_{REG} cells, and suppresses T_{H}17 cell responses during amelioration of experimental colitis and EAE [4,22]. Gut bacteria critically impact organ specific immune responses in experimental RA, T1D and EAE [23]. Therefore, the way gut bacteria influence the T_{REG}/T_{EFF} axis may be a critical component of human autoimmune diseases. Intriguingly, vitamin D has been shown to promote T_{REG} function and suppress T_{H}17 cells in mice and humans [36,37].

### Gut Bacteria, Vitamin D and EAE.

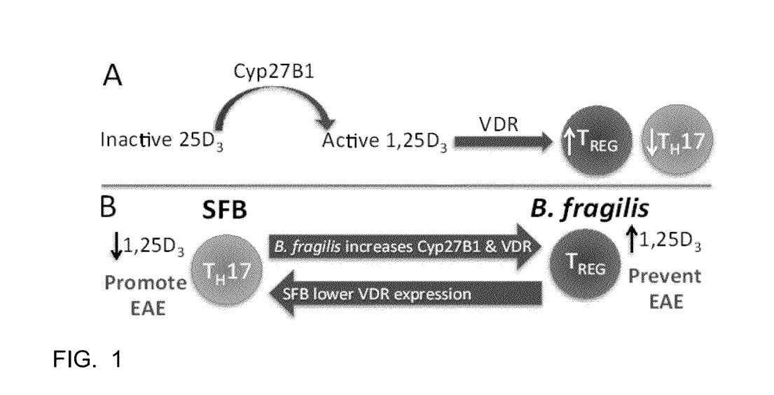

Studies are presented herein to support a fascinating and previously unexplored link between gut microbiota, vitamin D and experimental MS. The inventors recognize that both pathogenic (e.g., SFB) and protective (e.g., B. fragilis) bacteria in the gut may modulate the balance between T_{REG}/T_{EFF} functions by controlling the vitamin D system. Germ-free animals, born and raised under sterile conditions, display reduced incidence and severity of EAE [2,6,7]. Dendritic cells (DCs) from the gut of germ-free mice are reduced in their ability to prime T_{H}1 and T_{H}17 cell development, and gut colonization with SFB induces T_{H}1/T_{H}17 cells in the CNS resulting in EAE in germ-free mice [2]. As described herein, SFB colonization suppresses the vitamin D receptor (VDR), potentially promoting T_{H}1/T_{H}17 cells by inhibiting T_{REGS}. Remarkably, serum levels of active vitamin D are reduced in germ-free animals, along with colonic expression of the vitamin D converting enzyme, Cyp27b1. Colonization of germ-free mice with B. fragilis elevates serum vitamin D levels and increases Cyp27b1 expression in the colon; this activity requires PSA production by B. fragilis, and PSA treatment alone is sufficient to activate the vitamin D system. Further, as shown herein, EAE protection by PSA is diminished in mice on a vitamin D deficient diet, suggesting that PSA requires vitamin D for its beneficial functions. A proposed scheme to explain these data is presented in FIG. 1.

The inventors recognize that changes in the gut microbiota may be a feature of MS, although microbiome studies of MS patients have not yet been reported. Based on EAE data, vitamin D deficiency in MS patients may be related to changes in gut bacteria, as specific bacterial species may be required to regulate the vitamin D system. Thus, the inventors recognize that the gut microbiota may impact EAE via vitamin D-mediated modulation of immune responses, and provide a transformative treatment for MS.

### Germ-Free Mice are Attenuated in EAE Development

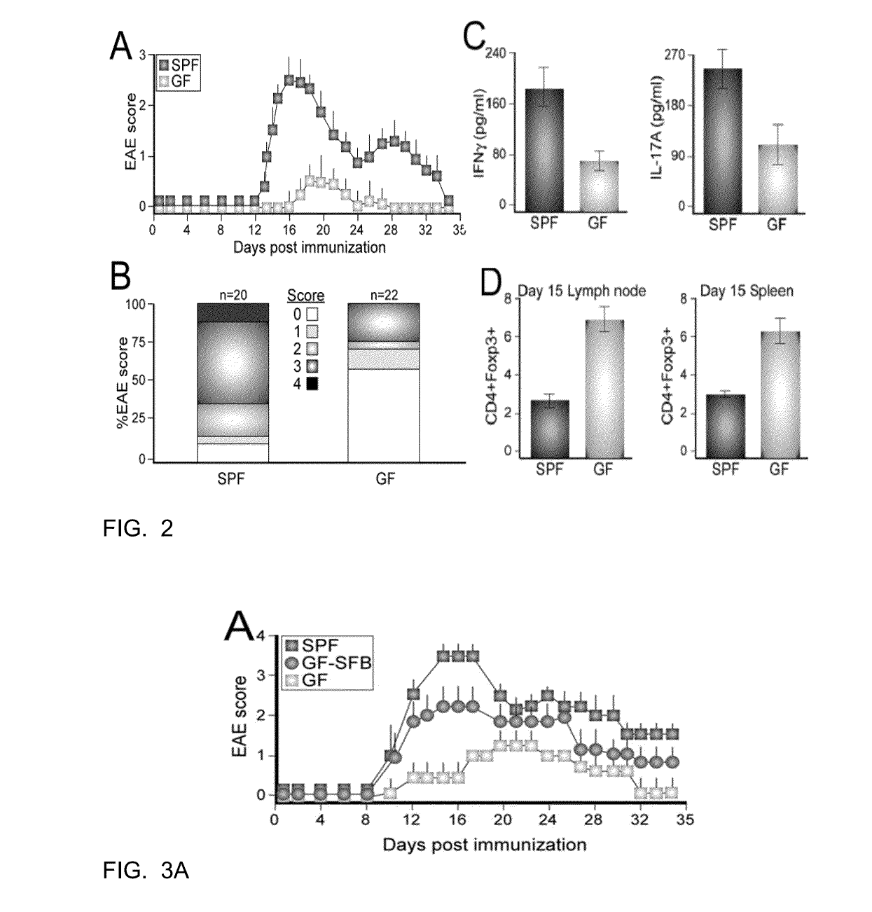

Although the contribution of the microbiota to GI function is well-documented, recent speculations propose that gut bacteria may control immune responses outside the gut [38,39]. The inventors reported that commensal microbes can influence extra-intestinal immunity by examining the role of the microbiota during EAE [2], and were the first to show that germ-free (GF) animals develop significantly reduced EAE symptoms compared to SPF (specific pathogen free) mice, as assessed by a standard scoring system (FIG. 2A). Disease incidence and severity are attenuated in GF mice (FIG. 2B). GF mice show reduced demyelination when examined histopathologically [2], and also express lower levels of IFNγ and IL17 (FIG. 2C), but display an increased proportion CD4+Foxp3+ T_{REGS} at the peak of disease (FIG. 2D). These data revealed that the microbiota modulates disease development and immune responses in EAE.

Intestinal Colonization with SFB Promotes EAE

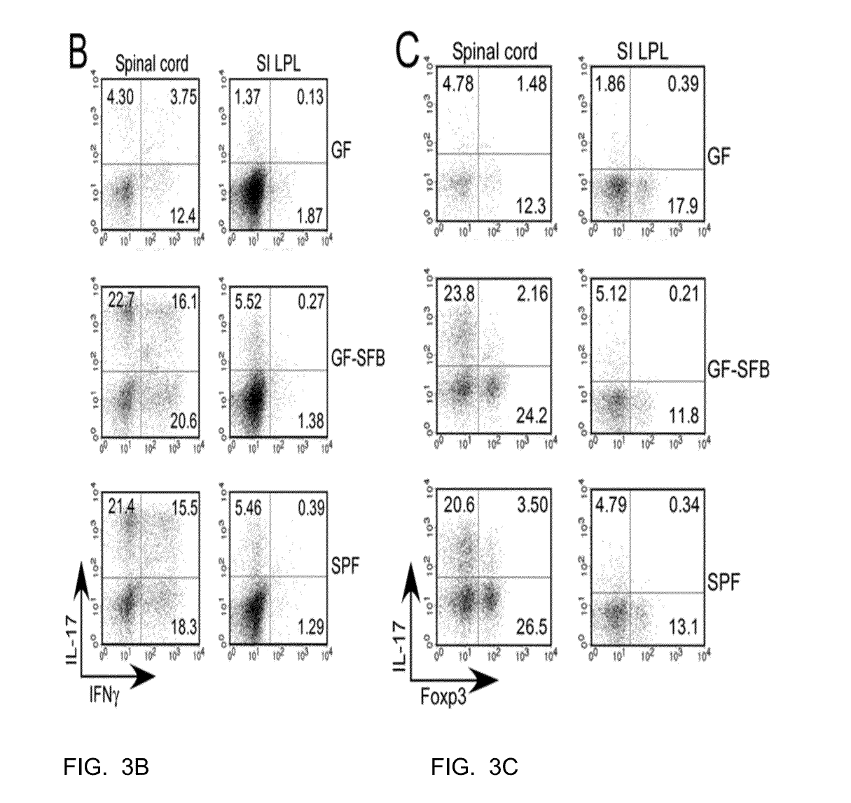

The attenuation of EAE in GF mice suggests that the microbiota stimulates pro-inflammatory immune responses that lead to inflammation and pathology in the CNS. T_{H}17 cells play a pivotal role in EAE; intriguingly, the microbiota is required for intestinal T_{H}17 cell development [40,41]. Recent studies have identified that Segmented Filamentous Bacteria (SFB) induce T_{H}17 cell differentiation in the gut of mice [42,43]. It was wondered if SFB were also able to promote T_{H}17 cells outside the gut, and perhaps restore the EAE phenotype in GF mice. Thus, groups of animals were either mono-colonized with SFB (GF-SFB) or left germ-free (GF), and compared to SPF mice. Remarkably, animals harboring intestinal SFB alone developed EAE (FIG. 3A). Although colonization with SFB did not induce EAE to the maximal level of SPF animals with a complex microbiota, GF-SFB animals showed pronounced disease compared to GF mice. Consistent with the findings that T_{H}1 and T_{H}17 cells are reduced in the spleens of GF animals (see FIG. 2C), a significant decrease in IL-17 producing CD4+ T cells in the small intestinal lamina propria (SI LPL) in the absence of a microbiota (compare GF and SPF) (FIG. 3B) was observed. Remarkably, CD4+ T cells harvested from the spinal cords of GF mice at the peak of disease also showed a decrease in IL-17 and IFNγ-producing single positive and double positive CD4+ T cells. Thus, the microbiota influences immune responses in the CNS. However, mono-association with SFB (GF-SFB) resulted in a considerable increase in pro-inflammatory IL-17 and IFNγ production in the spinal cords and intestines of mice. FIG. 3C shows that Foxp3+ T_{REGS} are increased in the SI LPL of GF mice, and mono-association with SFB reduces T_{REG} cells in the gut. Accordingly, colonization of GF animals with SFB resulted in a T_{H}17/T_{REG} profile in the spinal cord that was indistinguishable from SPF animals. These data show that the microbiota impacts EAE and the development of T_{H}1/T_{H}17 versus T_{REG} cells in the CNS.

### The Microbiota Regulates the Vitamin D System

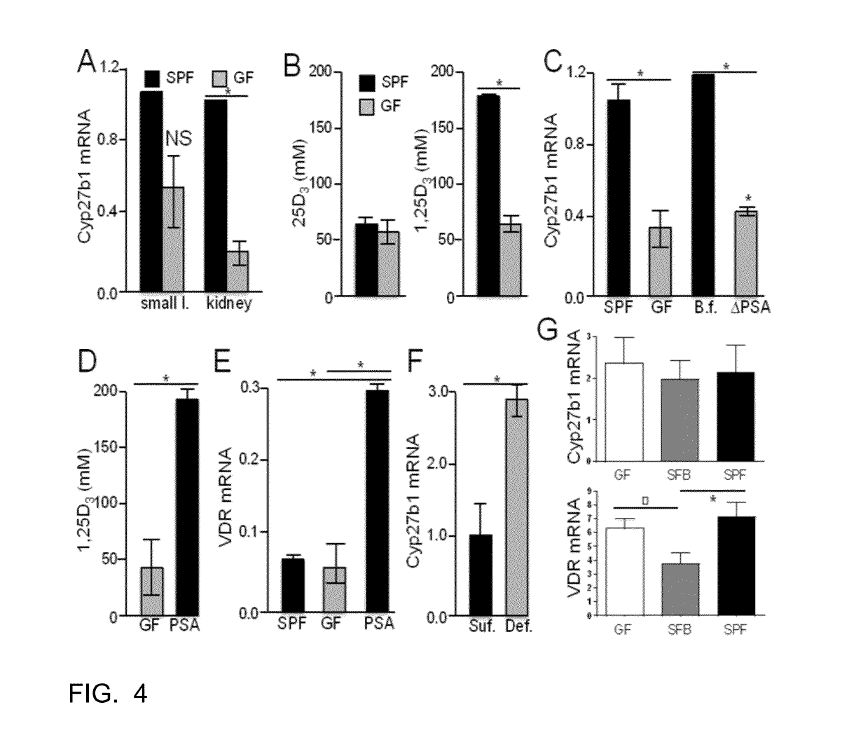

In addition to numerous studies showing that gut bacteria profoundly impact the immune system, the microbiota is linked to metabolic pathways such as vitamin metabolism [39]. Previous studies have documented expression of the vitamin D-activating enzyme, Cyp27b1, within its classical endocrine location in the kidney [44], as well as non-classical extra-renal sites such as the human colon [45]. To specifically assess the impact of gut bacteria to the vitamin D system in the gut, expression of Cyp27b1 and the vitamin D receptor (VDR) in GF mice was measured. Analysis of kidney tissue showed decreased mRNA levels of Cyp27b1 in GF mice (FIG. 4A). By contrast, there was no significant change in Cyp27b1 in the small intestine (FIG. 4A). However, dramatically reduced serum concentration of active 1,25D in GF mice represents a fascinating and novel finding linking gut bacteria to vitamin D status (FIG. 4B). Further, it is revealed that Cyp27b1 expression is lower in colonic tissues of GF mice (FIG. 4C; compare GF to SPF). Remarkably, mono-colonization of GF animals with B. fragilis restored Cyp27b1 expression in the colon to the levels found in SPF animals with a complex microbiota (FIG. 4C). This striking phenotype is dependent on PSA expression, as mono-colonization with B. fragilis lacking PSA (B. fragilisΔPSA) does not correct the decreased expression of Cyp27b1. Furthermore, oral feeding of PSA to GF mice increases serum levels of 1,25D (FIG. 4D). These data show that expression of the vitamin D converting enzyme is regulated by the microbiota, and specifically, that PSA induces colonic expression of Cyp27b1 and elevates circulating 1,25D levels. These findings suggest that elevation of Cyp27b1 expression by PSA leads to increased levels of serum 1,25D.

Vitamin D is sensed by VDR, which promotes gene expression in various cell types and tissues. Oral gavage of GF animals with PSA increases expression of VDR in the colon (FIG. 4E), consistent with its role in increasing the availability of active 1,25D. Most remarkably, PSA feeding induces increased colonic Cyp27b1 expression in mice on a vitamin D deficient diet (FIG. 4F), suggesting that PSA displays enhanced responsiveness during reduced vitamin D conditions (such as in MS patients).

To assess the impact of SFB on vitamin D responses, Cyp27b1 and VDR expression in GF, SFB mono-colonized and SPF mice were compared. mRNA levels in the small intestine (where SFB colonize) show that SFB do not induce Cyp27b1, however VDR expression is reduced (FIG. 4G). Thus, B. fragilis and SFB induce opposing T cell subsets (T_{REGS} vs. T_{H}17 cells), and have divergent impacts on vitamin D utilization. As vitamin D is associated with T_{REG} development [46], and vitamin D inhibits T_{H}17 cells [47], gut bacteria may modulate the T_{REG}/T_{H}17 axis by regulating the vitamin D system.

### Vitamin D Enhances PSA-Mediated Induction of T_{REG} Cells

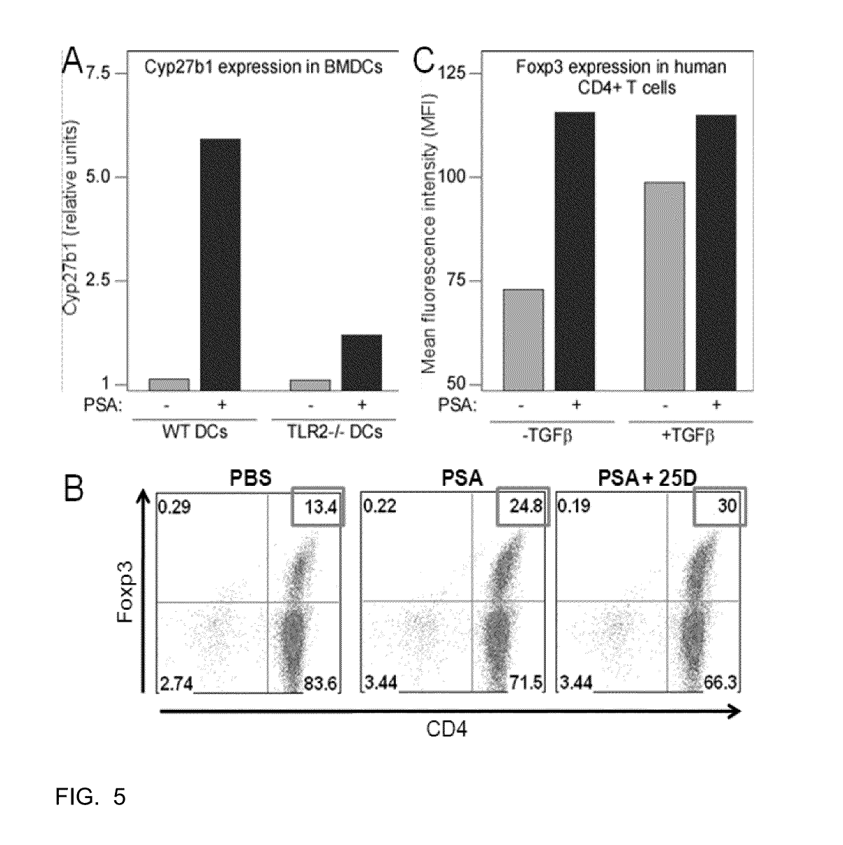

As described, the ability of PSA to protect against colitis & EAE in mice involves Foxp3+ T_{REGS} [8,48]. The inventors recognize that vitamin D may play a pivotal role in microbial-induced T_{REGS}, given the ability of 1,25D to promote T_{REG} generation [49] and the novel findings that PSA induces the vitamin D-system. First, the cell type that is responsible for PSA-dependent conversion of 25D to active 1,25D was made. The inventors have shown that PSA activates DCs, which prime T_{REG} responses [4,22]. Indeed, PSA induces Cyp27b1 expression from bone marrow-derived dendritic cells (BMDCs) (FIG. 5A). PSA requires Toll-like receptor 2 (TLR2) for T_{REG} induction [22]; Cyp27b1 expression required TLR2, as TLR2−/− DCs did not respond to PSA

Next tested was whether vitamin D enhanced the ability of PSA to induce Foxp3+ T_{REG} cells in vitro. BMDCs treated with PSA or PBS, with or without 25D, were incubated with CD4+ T cells from the spleens of mice. FIG. 5B shows that PSA increases the proportions of CD4+Foxp3+ T cells in culture, with this effect being enhanced by co-treatment with 25D. The Hewison group has described TLR-inducible expression of CYP27B1 in human DCs [50], and has shown the effects of vitamin D on human T_{REG} development in vitro [11]. However, the ability of PSA to induce human Foxp3+ T_{REG} cells has not been reported. It is now revealed that PSA promotes Foxp3 expression in human T_{REGS} in vitro (FIG. 5C). TGFβ (a known inducer of T_{REGS}) does not further enhance PSA-mediated T_{REG} cells, demonstrating the potency of PSA. These studies further link PSA-induced T_{REGS} to the vitamin D pathway. Thus, PSA and vitamin D may promote human T_{REG} cells.

PSA Protects Vitamin D Deficient Mice from EAE

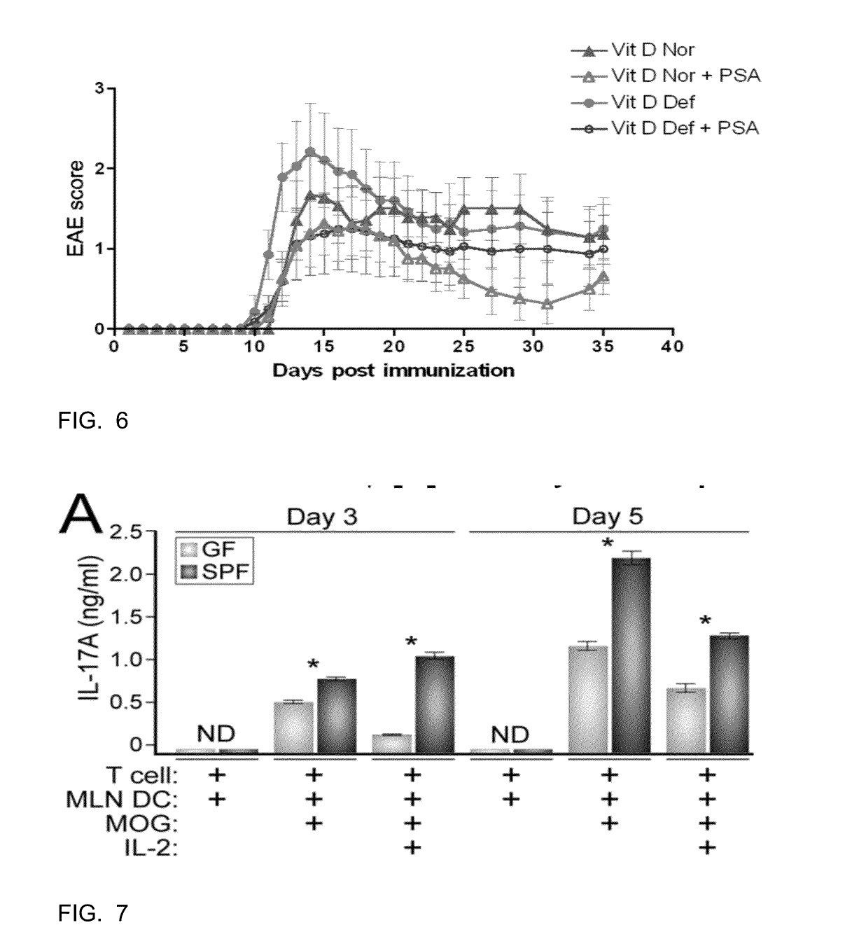

The data demonstrates that PSA activates the vitamin D system in vitro and in animals, and vitamin D enhances PSA-induced T_{REG} development in cell cultures. PSA protects mice from EAE [8]; however, it is unknown whether PSA protection requires vitamin D. To address whether PSA may serve as a viable therapy for vitamin D deficient MS patients, mice on vitamin D sufficient and deficient diets were raised, EAE was induced, and PSA's protective effects were assessed. Mice on a vitamin D deficient chow displayed worse EAE scores than animals on normal chow (FIG. 6), confirming a role for vitamin D in disease development. At the peak of disease (day 14), mice on both vitamin D sufficient and deficient diets were protected from EAE, demonstrating PSA activity in vitamin D deficient mice. Remarkably, PSA-treated mice were attenuated in disease during the remission phase if they were fed vitamin D sufficient diets (FIG. 6; compare PSA groups from day 20-35), showing PSA synergizes with vitamin D during protection from EAE. These data are the first to show a link between gut bacteria, vitamin D and EAE.

Environmental factors contribute to MS, and vitamin D deficiency is associated with an increased risk of disease. Based on data from EAE, gut bacteria may impact MS. Although vitamin D is being evaluated in the clinic, it may not be fully efficacious without also restoring gut bacteria that regulate vitamin D utilization. A combinatorial treatment with vitamin D and probiotics thus represents a transformative therapeutic approach for MS.

It has been shown that colonization of mice with SFB promotes EAE, but the molecular and cellular mechanisms by which gut bacteria enhance autoimmunity are unknown. Dendritic cells (DCs) are capable of priming various T-helper cell reactions in the gut [51]. Therefore, purified CD11c+ DCs from the mesenteric lymph nodes (MLNs) of GF and SPF animals were purified, and the cells co-cultured with MOG-specific T cells to measure IL-17 production in response to MOG peptide. Cytokine analysis revealed that DCs from GF mice were defective in promoting IL-17 expression when compared to DCs from SPF animals (FIG. 7A). Similar results were obtained for IFNγ [2]. No cytokine production was detected from co-cultures without MOG peptide stimulation, confirming antigen-specific T cell activation. Consequently, these results show that gut DCs from GF animals have a reduced capacity to induce both T_{H}1 and T_{H}17 cell response to self-antigens, consistent with reduced EAE development in animals without a microbiota

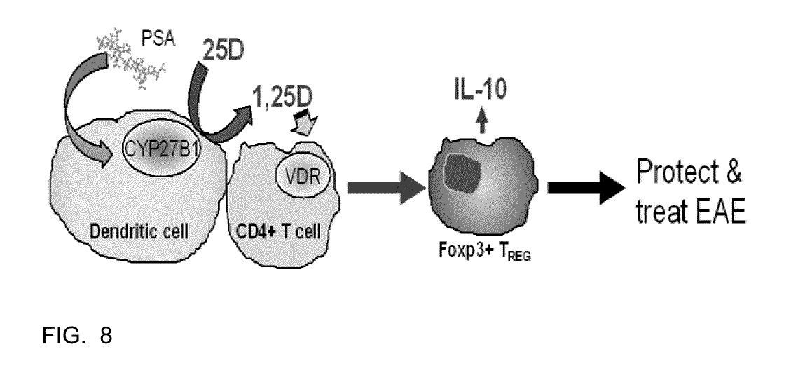

It is revealed for the first time that germ-free animals are deficient in expression of the vitamin D activating enzyme (Cyp27b1) and have reduced serum levels of active vitamin D (FIG. 4). PSA is a microbial molecule that increases expression of Cyp27b1 and VDR, increases active 1,25D, and protects from EAE (FIGS. 4 & 6). Furthermore, PSA's protective ability is enhanced by dietary vitamin D. These data indicate that PSA utilizes (and may require) vitamin D for induction of T_{REGS} during protection from EAE. An explanation of the novel findings is provided in FIG. 8.

### REFERENCES

The following publications are incorporated by reference herein:
1. Kakalacheva K, Lunemann J D (2011) Environmental triggers of multiple sclerosis. FEBS letters 585: 3724-3729.
2. Lee Y K, Menezes J S, Umesaki Y, Mazmanian S K (2011) Proinflammatory T-cell responses to gut microbiota promote experimental autoimmune encephalomyelitis. Proc Natl Acad Sci USA 108 Suppl 1: 4615-4622.
3. Mazmanian S K, Liu C H, Tzianabos A O, Kasper D L (2005) An immunomodulatory molecule of symbiotic bacteria directs maturation of the host immune system. Cell 122: 107-118.
4. Mazmanian S K, Round J L, Kasper D L (2008) A microbial symbiosis factor prevents intestinal inflammatory disease. Nature 453: 620-625.
5. Round J L, Lee S M, Li J, Tran G, Jabri B, et al. (2011) The Toll-like receptor 2 pathway establishes colonization by a commensal of the human microbiota. Science 332: 974-977.
6. Ochoa-Reparaz J, Mielcarz D W, Ditrio L E, Burroughs A R, Foureau D M, et al. (2009) Role of gut commensal microflora in the development of experimental autoimmune encephalomyelitis. J Immunol 183: 6041-6050.
7. Berer K, Mues M, Koutrolos M, Rasbi Z A, Boziki M, et al. (2011) Commensal microbiota and myelin autoantigen cooperate to trigger autoimmune demyelination. Nature 479: 538-541.
8. Ochoa-Reparaz J, Mielcarz D W, Wang Y, Begum-Haque S, Dasgupta S, et al. (2010) A polysaccharide from the human commensal Bacteroides fragilis protects against CNS demyelinating disease. Mucosal Immunol 3: 487-495.
9. Ochoa-Reparaz J, Mielcarz D W, Ditrio L E, Burroughs A R, Begum-Haque S, et al. (2010) Central nervous system demyelinating disease protection by the human commensal Bacteroides fragilis depends on polysaccharide A expression. J Immunol 185: 4101-4108.
10. Adams J S, Hewison M (2008) Unexpected actions of vitamin D: new perspectives on the regulation of innate and adaptive immunity. Nat Clin Pract Endocrinol Metab 4: 80-90.
11. Jeffery L E, Burke F, Mura M, Zheng Y, Qureshi O S, et al. (2009) 1,25-Dihydroxyvitamin D(3) and IL-2 combine to inhibit T cell production of inflammatory cytokines and promote development of regulatory T cells expressing CTLA-4 and FoxP3. J Immunol 183: 5458-5467.
12. Goverman J (2009) Autoimmune T cell responses in the central nervous system. Nat Rev Immunol 9: 393-407.
13. Bhat R, Steinman L (2009) Innate and adaptive autoimmunity directed to the central nervous system. Neuron 64: 123-132.
14. Becker K G, Simon R M, Bailey-Wilson J E, Freidlin B, Biddison W E, et al. (1998) Clustering of non-major histocompatibility complex susceptibility candidate loci in human autoimmune diseases. Proc Natl Acad Sci USA 95: 9979-9984.
15. Willer C J, Dyment D A, Risch N J, Sadovnick A D, Ebers G C (2003) Twin concordance and sibling recurrence rates in multiple sclerosis. Proc Natl Acad Sci USA 100: 12877-12882.
16. Baranzini S E, Mudge J, van Velkinburgh J C, Khankhanian P, Khrebtukova I, et al. (2010) Genome, epigenome and RNA sequences of monozygotic twins discordant for multiple sclerosis. Nature 464: 1351-1356.
17. Bach J F (2002) The effect of infections on susceptibility to autoimmune and allergic diseases. N Engl J Med 347: 911-920.
18. Stromnes I M, Goverman J M (2006) Active induction of experimental allergic encephalomyelitis. Nat Protoc 1: 1810-1819.
19. Kakalacheva K, Munz C, Lunemann J D (2011) Viral triggers of multiple sclerosis. Biochimica et biophysica acta 1812: 132-140.
20. Ley R E, Peterson D A, Gordon J I (2006) Ecological and evolutionary forces shaping microbial diversity in the human intestine. Cell 124: 837-848.
21. Round J L, Mazmanian S K (2009) The gut microbiota shapes intestinal immune responses during health and disease. Nat Rev Immunol.
22. Round J L, Mazmanian S K (2010) Inducible Foxp3+ regulatory T-cell development by a commensal bacterium of the intestinal microbiota. Proc Natl Acad Sci USA 107: 12204-12209.
23. Round J L, O'Connell R M, Mazmanian S K (2009) Coordination of tolerogenic immune responses by the commensal microbiota. J Autoimmun.
24. Runia T F, Hop W C, de Rijke Y B, Buljevac D, Hintzen R Q (2012) Lower serum vitamin D levels are associated with a higher relapse risk in multiple sclerosis. Neurology 79: 261-266.
25. Stewart N, Simpson S, Jr., van der Mei I, Ponsonby A L, Blizzard L, et al. (2012) Interferon-beta and serum 25-hydroxyvitamin D interact to modulate relapse risk in MS. Neurology 79: 254-260.
26. Raghuwanshi A, Joshi S S, Christakos S (2008) Vitamin D and multiple sclerosis. J Cell Biochem 105: 338-343.
27. Pierrot-Deseilligny C, Souberbielle J C Is hypovitaminosis D one of the environmental risk factors for multiple sclerosis? Brain 133: 1869-1888.
28. Ascherio A, Munger K L, Simon K C Vitamin D and multiple sclerosis. Lancet Neurol 9: 599-612.
29. Solomon A J, Whitham R H Multiple sclerosis and vitamin D: a review and recommendations. Curr Neurol Neurosci Rep 10: 389-396.
30. Spach K M, Hayes C E (2005) Vitamin D3 confers protection from autoimmune encephalomyelitis only in female mice. J Immunol 175: 4119-4126.
31. Spach K M, Pedersen L B, Nashold F E, Kayo T, Yandell B S, et al. (2004) Gene expression analysis suggests that 1,25-dihydroxyvitamin D3 reverses experimental autoimmune encephalomyelitis by stimulating inflammatory cell apoptosis. Physiol Genomics 18: 141-151.
32. Pedersen L B, Nashold F E, Spach K M, Hayes C E (2007) 1,25-dihydroxyvitamin D3 reverses experimental autoimmune encephalomyelitis by inhibiting chemokine synthesis and monocyte trafficking J Neurosci Res 85: 2480-2490.
33. Sakaguchi S, Yamaguchi T, Nomura T, Ono M (2008) Regulatory T cells and immune tolerance. Cell 133: 775-787.
34. Nylander A, Hafler D A (2012) Multiple sclerosis. The Journal of clinical investigation 122: 1180-1188.
35. Chow J, Mazmanian S K (2009) Getting the bugs out of the immune system: do bacterial microbiota “fix” intestinal T cell responses? Cell Host Microbe 5: 8-12.
36. Chang J H, Cha H R, Lee D S, Seo K Y, Kweon M N (2010) 1,25-Dihydroxyvitamin D3 inhibits the differentiation and migration of T(H)17 cells to protect against experimental autoimmune encephalomyelitis. PLoS One 5: e12925.
37. Allen A C, Kelly S, Basdeo S A, Kinsella K, Mulready K J, et al. (2012) A pilot study of the immunological effects of high-dose vitamin D in healthy volunteers. Multiple sclerosis.
38. Noverr M C, Huffnagle G B (2004) Does the microbiota regulate immune responses outside the gut? Trends Microbiol 12: 562-568.
39. Lee Y K, Mazmanian S K (2010) Has the microbiota played a critical role in the evolution of the adaptive immune system? Science 330: 1768-1773.
40. Atarashi K, Nishimura J, Shima T, Umesaki Y, Yamamoto M, et al. (2008) ATP drives lamina propria T(H)17 cell differentiation. Nature 455: 808-812.
41. Ivanov, I I, Frutos Rde L, Manel N, Yoshinaga K, Rifkin D B, et al. (2008) Specific microbiota direct the differentiation of IL-17-producing T-helper cells in the mucosa of the small intestine. Cell Host Microbe 4: 337-349.
42. Gaboriau-Routhiau V, Rakotobe S, Lecuyer E, Mulder I, Lan A, et al. (2009) The key role of segmented filamentous bacteria in the coordinated maturation of gut helper T cell responses. Immunity 31: 677-689.
43. Ivanov, I I, Atarashi K, Manel N, Brodie E L, Shima T, et al. (2009) Induction of intestinal Th17 cells by segmented filamentous bacteria. Cell 139: 485-498.
44. Zehnder D, Bland R, Walker E A, Bradwell A R, Howie A J, et al. (1999) Expression of 25-hydroxyvitamin D3-1alpha-hydroxylase in the human kidney. J Am Soc Nephrol 10: 2465-2473.
45. Zehnder D, Bland R, Williams M C, McNinch R W, Howie A J, et al. (2001) Extrarenal expression of 25-hydroxyvitamin d(3)-1 alpha-hydroxylase. J Clin Endocrinol Metab 86: 888-894.
46. Chambers E S, Hawrylowicz C M (2011) The impact of vitamin D on regulatory T cells. Curr Allergy Asthma Rep 11: 29-36.
47. Palmer M T, Lee Y K, Maynard C L, Oliver J R, Bikle D D, et al. (2011) Lineage-specific effects of 1,25-dihydroxyvitamin D(3) on the development of effector CD4 T cells. The Journal of biological chemistry 286: 997-1004.
48. Round J L, Mazmanian S K Inducible Foxp3+ regulatory T-cell development by a commensal bacterium of the intestinal microbiota. Proc Natl Acad Sci USA 107: 12204-12209.
49. Barrat F J, Cua D J, Boonstra A, Richards D F, Crain C, et al. (2002) In vitro generation of interleukin 10-producing regulatory CD4(+) T cells is induced by immunosuppressive drugs and inhibited by T helper type 1 (Th1)- and Th2-inducing cytokines J Exp Med 195: 603-616.
50. Hewison M, Freeman L, Hughes S V, Evans K N, Bland R, et al. (2003) Differential regulation of vitamin D receptor and its ligand in human monocyte-derived dendritic cells. J Immunol 170: 5382-5390.
51. Coombes J L, Maloy K J (2007) Control of intestinal homeostasis by regulatory T cells and dendritic cells. Semin Immunol 19: 116-126.
52. Wu H J, Ivanov, I I, Darce J, Hattori K, Shima T, et al. (2010) Gut-residing segmented filamentous bacteria drive autoimmune arthritis via T helper 17 cells. Immunity 32: 815-827.
53. Bar-On L, Jung S (2010) Defining in vivo dendritic cell functions using CD11c-DTR transgenic mice. Methods in molecular biology 595: 429-442.
54. Bruce D, Yu S, Ooi J H, Cantorna M T (2011) Converging pathways lead to overproduction of IL-17 in the absence of vitamin D signaling. International immunology 23: 519-528.
55. Joshi S, Pantalena L C, Liu X K, Gaffen S L, Liu H, et al. (2011) 1,25-dihydroxyvitamin D(3) ameliorates Th17 autoimmunity via transcriptional modulation of interleukin-17A. Molecular and cellular biology 31: 3653-3669.
56. Isaksson M, Lundgren B A, Ahlgren K M, Kampe O, Lobell A (2012) Conditional DC depletion does not affect priming of encephalitogenic Th cells in EAE. European journal of immunology.
57. Krutzik S R, Hewison M, Liu P T, Robles J A, Stenger S, et al. (2008) IL-15 links TLR2/1-induced macrophage differentiation to the vitamin D-dependent antimicrobial pathway. J Immunol 181: 7115-7120.
58. Cabrera R, Ararat M, Eksioglu E A, Cao M, Xu Y, et al. (2010) Influence of serum and soluble CD25 (sCD25) on regulatory and effector T-cell function in hepatocellular carcinoma. Scandinavian journal of immunology 72: 293-301.
59. Maynard C L, Hatton R D, Helms W S, Oliver J R, Stephensen C B, et al. (2009) Contrasting roles for all-trans retinoic acid in TGF-beta-mediated induction of Foxp3 and I110 genes in developing regulatory T cells. The Journal of experimental medicine 206: 343-357.
60. Adams J S, Hewison M (2012) Extrarenal expression of the 25-hydroxyvitamin D-1-hydroxylase. Archives of biochemistry and biophysics 523: 95-102.
61. Correale J, Ysrraelit M C, Gaitan M I (2011) Vitamin D-mediated immune regulation in multiple sclerosis. Journal of the neurological sciences 311: 23-31.
62. Mayne C G, Spanier J A, Relland L M, Williams C B, Hayes C E (2011) 1,25-Dihydroxyvitamin D3 acts directly on the T lymphocyte vitamin D receptor to inhibit experimental autoimmune encephalomyelitis. European journal of immunology 41: 822-832.
63. Sigmundsdottir H, Pan J, Debes G F, Alt C, Habtezion A, et al. (2007) DCs metabolize sunlight-induced vitamin D3 to ‘program’ T cell attraction to the epidermal chemokine CCL27. Nature immunology 8: 285-293.
64. Liu P T, Stenger S, Li H, Wenzel L, Tan B H, et al. (2006) Toll-like receptor triggering of a vitamin D-mediated human antimicrobial response. Science 311: 1770-1773.
65. Power C, Antony J M, Ellestad K K, Deslauriers A, Bhat R, et al. (2010) The human microbiome in multiple sclerosis: pathogenic or protective constituents? The Canadian journal of neurological sciences Le journal canadien des sciences neurologiques 37 Suppl 2: S24-33.
66. Berer K, Krishnamoorthy G (2012) Commensal gut flora and brain autoimmunity: a love or hate affair? Acta neuropathologica 123: 639-651.
67. Goverman J, Woods A, Larson L, Weiner L P, Hood L, et al. (1993) Transgenic mice that express a myelin basic protein-specific T cell receptor develop spontaneous autoimmunity. Cell 72: 551-560.
68. Bettelli E, Pagany M, Weiner H L, Linington C, Sobel R A, et al. (2003) Myelin oligodendrocyte glycoprotein-specific T cell receptor transgenic mice develop spontaneous autoimmune optic neuritis. The Journal of experimental medicine 197: 1073-1081.
69. Cantorna M T, Hayes C E, DeLuca H F (1996) 1,25-Dihydroxyvitamin D3 reversibly blocks the progression of relapsing encephalomyelitis, a model of multiple sclerosis. Proceedings of the National Academy of Sciences of the United States of America 93: 7861-7864.
70. Jeffery L E, Burke F, Mura M, Zheng Y, Qureshi O S, et al. (2009) 1,25-Dihydroxyvitamin D3 and IL-2 combine to inhibit T cell production of inflammatory cytokines and promote development of regulatory T cells expressing CTLA-4 and FoxP3. J Immunol 183: 5458-5467.
71. Morales-Tirado V, Wichlan D G, Leimig T E, Street S E, Kasow K A, et al. (2011) 1alpha,25-dihydroxyvitamin D3 (vitamin D3) catalyzes suppressive activity on human natural regulatory T cells, uniquely modulates cell cycle progression, and augments FOXP3. Clinical immunology 138: 212-221.
72. McMurchy A N, Levings M K (2012) Suppression assays with human T regulatory cells: a technical guide. European journal of immunology 42: 27-34.
73. Baecher-Allan C M, Costantino C M, Cvetanovich G L, Ashley C W, Beriou G, et al. (2011) CD2 costimulation reveals defective activity by human CD4+CD25(hi) regulatory cells in patients with multiple sclerosis. Journal of immunology 186: 3317-3326.
74. Anderson A C, Chandwaskar R, Lee D H, Sullivan J M, Solomon A, et al. (2012) A transgenic model of central nervous system autoimmunity mediated by CD4+ and CD8+ T and B cells. Journal of immunology 188: 2084-2092.
75. Tzianabos A O, et al. (1992) The capsular polysaccharide of Bacteriodes fragilis comprises two ionically linked polysaccharides, Journal of Biological Chemistry, 267:18230-18235

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A method for treating multiple sclerosis in a subject in need of such treatment, comprising administering a) polysaccharide A and b) vitamin D or a vitamin D metabolite to the subject in an amount effective to treat multiple sclerosis.

2. The method of claim 1, wherein administering polysaccharide A comprises administering B. fragilis cells containing polysaccharide A.

3. The method of claim 1, wherein administering polysaccharide A comprises administering purified polysaccharide A.

4. The method of claim 1, wherein the vitamin D metabolite is 25-hydroxyvitamin D₃ or 1,25-dihydroxyvitamin D₃.

5. The method of claim 1, wherein the subject is a mouse or a human.

6. The method of claim 1, wherein the polysaccharide A and the vitamin D or a vitamin D metabolite are administered orally.

7. A method for increasing T_{REG} cells in a subject, comprising administering a) polysaccharide A and b) vitamin D or a vitamin D metabolite to the subject in an amount effective to increase the number of T_{REG} cells in the central nervous system of the subject.

8. The method of claim 7, wherein administering polysaccharide A comprises administering B. fragilis cells containing polysaccharide A.

9. The method of claim 7, wherein administering polysaccharide A comprises administering purified polysaccharide A.

10. The method of claim 7, wherein the vitamin D metabolite is 25-hydroxyvitamin D₃ or 1,25-dihydroxyvitamin D₃.

11. The method of claim 7, wherein the subject is a mouse or a human.

12. The method of claim 7, wherein the polysaccharide A and the vitamin D or a vitamin D metabolite are administered orally.
